(19) United States
(12) Patent Application Publication
(10) Pub. No.: US 2009/0069623 A1
(43) Pub. Date: Mar. 12, 2009

(54) METHODS AND COMPOSITIONS FOR TREATMENT OR PREVENTION OF RADIATION-INDUCED FIBROSIS

(75) Inventor: YOUNGMAN OH, Glen Allen, VA (US)

Correspondence Address: TROUTMAN SANDERS LLP, 600 PEACHTREE STREET , NE, ATLANTA, GA, 30308, US

(73) Assignee: Virginia Commonwealth University Intellectual Property Foundation, Richmond, VA (US)

(21) Appl. No.: 12/195,845

(22) Filed: Aug. 21, 2008

Related U.S. Application Data
(60) Provisional application No. 60/956,999, filed on Aug. 21, 2007.

(51) Int. Cl. A61B 6/00; A61K 31/404; A61K 31/366; A61K 31/40; A61K 31/435; A61K 31/47; A61K 39/395; A61K 38/00; A61K 31/7088

(52) U.S. Cl. 600/1; 514/419; 514/460; 514/423; 514/277; 514/311; 424/172.1; 424/133.1; 514/2; 514/44

(57) ABSTRACT

The present invention comprises methods and compositions for the treatment or prevention of radiation-induced fibrosis. Methods and compositions for the inhibition of CTGF are disclosed herein. Methods and compositions for treatment of neoplastic disease are disclosed herein. Inhibition of CTGF in humans or animals that have been exposed to ionizing radiation results in treatment or prevention of radiation-induced fibrosis.

### CROSS REFERENCE TO RELATED PATENT APPLICATIONS

The present invention claims the priority of U.S. Provisional Patent Application Ser. No. 60/956,999, filed Aug. 21, 2007, which is herein incorporated in its entirety.

### TECHNICAL FIELD

The present invention is related to methods and compositions for treatment or prevention of radiation-induced fibrosis. In particular, the invention is related to compositions and methods affecting CTGF.

### BACKGROUND OF THE INVENTION

Treatment of tumors by radiation therapy creates a dilemma common to many cancer treatments. A sufficient amount of treatment agent, for example, radiation rate, must be provided to the patient to treat and overcome the tumor, but the amount of the treatment agent has to be limited so that normal tissue is not injured to a great extent. Often in radiation therapy, there are side effects, many of which are dependent on the area of the body that is irradiated. A side effect seen with ionizing radiation is radiation-induced fibrosis in the area of the body that was irradiated.

Radiation-induced fibrosis (RIF) remains the most important dose-limiting toxicity of radiation therapy to soft tissue. RIF can develop as a late effect of radiation therapy in skin and subcutaneous tissue, lungs, the gastrointestinal and genitourinary tracts, muscles, or other organs, depending upon the treatment site. RIF may cause both cosmetic and functional impairment, which can lead to death or a significant deterioration in the quality of life. Functionally, RIF is reflected in loss of range of motion and muscle strength and the development of limb edema and pain.

The severity and development of RIF is influenced by multiple factors, including the radiation dose and volume, fractionation schedule, previous or concurrent treatments, genetic susceptibility, and co-morbidities, such as diabetes mellitus. Unlike a trauma-induced wound, where platelets and other cells from the circulation system migrate into the injured tissue, and repair is accomplished without fibrosis, high dose radiation induces cellular damage that results in cellular changes that inevitably leads to dysfunctional repair and RIF, if not ulceration and necrosis.

Classic theories of radiation effects indicate that DNA double-strand breaks are an early lethal event; however, the causes of RIF are not explained by slow cell turn-over or delayed cell death because late radiation toxicity, such as RIF, may be seen over a decade after irradiation. Although RIF originally was assumed to be a slow, irreversible process, recent studies suggest that RIF may not be a fixed process. Thus, there have been many theories, but few to no therapies, for treatment or prevention of RIF. The prevention of RIF has focused on improvements in radiation techniques, which have resulted in higher doses to the tumor target and decreased doses to normal tissue, thus limiting the development of RIF.

Radiation-induced pulmonary injury is an example of the extent of damage caused by RIF after treatment for thoracic or lung cancer. For patients treated for lung cancer, approximately 5-20% develop symptomatic lung injury, 50-100% develop radiologic evidence of regional injury, and 50-90% experience declines in pulmonary function. Radiation-induced pulmonary injury is a major limiting factor in the successful treatment of thoracic tumors. Similar levels of impairment are seen with radiation treatment of other body areas.

What is needed are methods and compositions that are provided to patients who are undergoing or have undergone radiation treatments for the prevention and treatment of radiation-induced fibrosis. What is also needed are convenient dosage formulas to enable patient compliance with such methods and treatment regimens.

### SUMMARY

The present invention comprises methods and compositions for the treatment or prevention of radiation-induced fibrosis and the related sequellae resulting from irradiation of human or animal bodies with ionizing radiation. In one aspect, the present invention comprises methods comprising administering an effective amount of compositions for the treatment or prevention of radiation-induced fibrosis (RIF) in humans and animals. One aspect of the invention comprises methods for inhibiting cellular cytokines, e.g., CTGF, that are involved in the formation of RIF, and thus, treating or preventing RIF.

A method of the present invention comprises providing compositions that inhibit an activity of a cytokine known as CTGF, IGFBP-8 or CCN2, hereinafter referred to as CTGF (connective tissue growth factor). CTGF is a cysteine-rich protein with a molecular weight of 36-38 kDa. IGFBP-8 is insulin-like growth factor binding protein-8, and CCN2 is an alternate name for CTGF indicating it as a member of the CCN family, which stands for CTGF, CEF10/Cyr61, and Nov (CCN).

Methods of the present invention comprise administering an effective amount of a composition comprising an inhibitor of CTGF to a human or animal to prevent or treat RIF. Inhibitors of CTGF include, but are not limited to 3-hydroxy-3-methylglutaryl coenzyme A (HMG-CoA) reductase inhibitors, such as statins, antisense polynucleotides, antibodies, RNA interference molecules, among others. Known HMG-CoA reductase inhibitors include, but are not limited to, statins, including lovastatin, simvastatin, pravastatin, fluvastatin, atorvastatin, mevastatin, rivastatin, cer(i)vastatin, pitavastatin, nisvastatin, itavastatin, rosuvastatin, and visastatin.

Methods of the present invention comprise in vitro cell systems for determining whether a compound or composition is an inhibitor of CTGF. Methods for testing compounds that inhibit CTGF are disclosed herein. Methods for identifying compounds or compositions that are inhibitors of CTGF comprise screening for compounds or compositions that inhibit CTGF, such as those compounds or compositions that inhibit CTGF in a manner similar or dissimilar to CTGF inhibitors, such as an HMG-CoA reductase inhibitor compound.

Methods of the present invention comprise treating or preventing a disorder associated with CTGF activity or the presence of CTGF by inhibiting CTGF expression or activity by administering a compound or composition that inhibits CTGF expression or activity. Methods of the present invention comprise treating a radiation-exposed individual or preventing tissue injury in an individual who is currently or subsequently exposed to ionizing radiation comprising administering to the individual, human or animal, an effective amount of a compound or composition that inhibits CTGF. Such compound or composition may comprise one or more HMG-CoA reductase inhibitors.

Methods of the present invention comprise treating a patient with a neoplastic disease, comprising administering to the patient an effective amount of a CTGF inhibitor, including but not limited to, an HMG-CoA reductase inhibitor, and treating the patient with radiation therapy.

### DESCRIPTION OF THE FIGURES

FIG. 1 is a Western blot of radiation-induced CTGF expression in HFL-1 cells.

FIG. 2 is a Western blot of radiation-induced CTGF expression in human lung cancer cells.

FIG. 3 is a Western blot of radiation-induced CTGF expression in M12 human prostate cancer cells.

FIG. 4 is a Western blot of demonstrating the effect of statin compounds on radiation-induced CTGF expression in HLF-1 cells.

FIG. 5A is an agarose gel of an RT-PCR analysis of mRNA expression in HFL-1 cells treated with TGF-β1.

FIG. 5B is a Western blot of HFL-1 cells treated with TGF-β1.

FIG. 6A is an agarose gel of an RT-PCR analysis of mRNA expression in irradiated HFL-1 cells.

FIG. 6B is a Western blot of irradiated HFL-1 cells.

FIG. 6C is a Western blot of HFL-1 cells exposed to 5 Gy of radiation.

FIG. 7A is an agarose gel of an RT-PCR analysis of mRNA expression in HFL-1 cells treated with TGF-β1 and simvastatin.

FIG. 7B is a Western blot of HFL-1 cells treated with TGF-β1 and simvastatin.

FIG. 8A is an agarose gel of an RT-PCR analysis of mRNA expression in irradiated HFL-1 cells treated with simvastatin.

FIG. 8B is a Western blot of irradiated HFL-1 cells treated with simvastatin.

FIG. 8C is a Western blot of irradiated HFL-1 cells treated with HMG-CoA inhibitors.

FIG. 9A is an agarose gel of an RT-PCR analysis of mRNA expression in HDF cells treated with TGF-β1.

FIG. 9B is a Western blot of HDF cells treated with TGF-β1.

FIG. 10A is an agarose gel of an RT-PCR analysis of mRNA expression in HDF cells treated with TGF-β1 and simvastatin.

FIG. 10B is a Western blot of HDF cells treated with TGF-β1 and simvastatin.

FIG. 11 is a Western blot of irradiated HDF cells.

### DETAILED DESCRIPTION

The present invention comprises methods and compositions for the treatment, prevention, or amelioration of disease by inhibiting CTGF. The present invention comprises methods and compositions for the treatment, prevention, or amelioration of radiation-induced fibrosis (RIF) by inhibiting CTGF associated with radiation therapies.

RIF is an after-effect of exposure of a human or animal body to ionizing radiation. Thoracic radiation effects are discussed herein as an example, but are not intended to limit the invention. Radiation induced lung injury is the main dose-limiting factor when irradiating the lung, for example, for treatment of lung cancer, tumors or neoplastic disease such as cancer, Hodgkin's lymphoma or non-Hodgkin's lymphoma. As such, organ or tissue tolerance limits the therapeutic options for treatment of cancer or neoplastic disease.

The etiology and cellular factors associated with RIF are unclear, prior to the present invention. Some studies have suggested that RIF might be related to the presence of TGF-β, which has been shown to be upregulated by radiation therapy. Other studies of RIF have shown no association with TGF-β. In some pathways, CTGF has been seen as a downstream effector of TGF-β, and though not wishing to be bound by any particular theory, it is theorized that CTGF may be involved in RIF. Thus, it is theorized that suppression of radiation-induced CTGF may prevent RIF in the lung, and in other organs affected by ionizing radiation, such as the lungs, kidneys, intestines, bladder, skin, and other body structures. Although the advent of more sophisticated radiation therapy techniques, such as three dimensional conformal radiation therapy or intensity-modulated radiation therapy, can permit dose escalation by limiting the normal tissue complication probability, RIF has not been eliminated, and therapy for, or prevention of, RIF presents a continuing problem.

For example, RIF in the lung may manifest as two distinct, though potentially connected, abnormalities. One manifestation is radiation pneumonitis, which is an early inflammatory reaction involving alveolar cell depletion and inflammatory cell accumulation in the interstitial space that occurs within 12 weeks after lung radiation therapy. The second manifestation is a late phase of RIF, considered until recently as irreversible, that consists mainly of fibroblast proliferation, collagen accumulation, and destruction of the normal lung architecture. Although studies have attempted to elucidate the mechanisms leading to RIF, the pathogenesis of RIF lung injury, and other RIF injury, at the cellular and molecular level is still incompletely described.

Differing results are seen in published accounts of the role of CTGF in RIF. CTGF has been shown to be a regulator of fibroblast proliferation, cell adhesion, and the stimulation of extracellular matrix production. Studies have shown that CTGF plays a role in the pathogenesis of fibrotic disorders, such as idiopathic pulmonary fibrosis, scleroderma, diabetic nephropathy, glomerulosclerosis, cirrhosis, and diabetic retinopathy. Recent data indicate that CTGF can be produced in a TGF-β independent manner and induce fibrosis in fibroblasts, indicating several pathways may exist for induction of CTGF. Some studies have shown that subcutaneous injection of TGF-β into neonatal rats results only in a transient fibrotic response, whereas co-injection of CTGF and TGF-β results in sustained fibrosis, suggesting that TGF-β may initiate a fibrotic incidence, but CTGF may be needed to sustain the fibrotic response. Other studies have shown that blocking CTGF expression using a specific siRNA or neutralizing antibodies results in suppression of fibrotic proteins, such as fibronectin and collagens, and may inhibit the fibrotic response in systemic sclerosis, liver fibrosis, and idiopathic pulmonary fibrosis; however, the role of CTGF in radiation-induced fibrosis was not known.

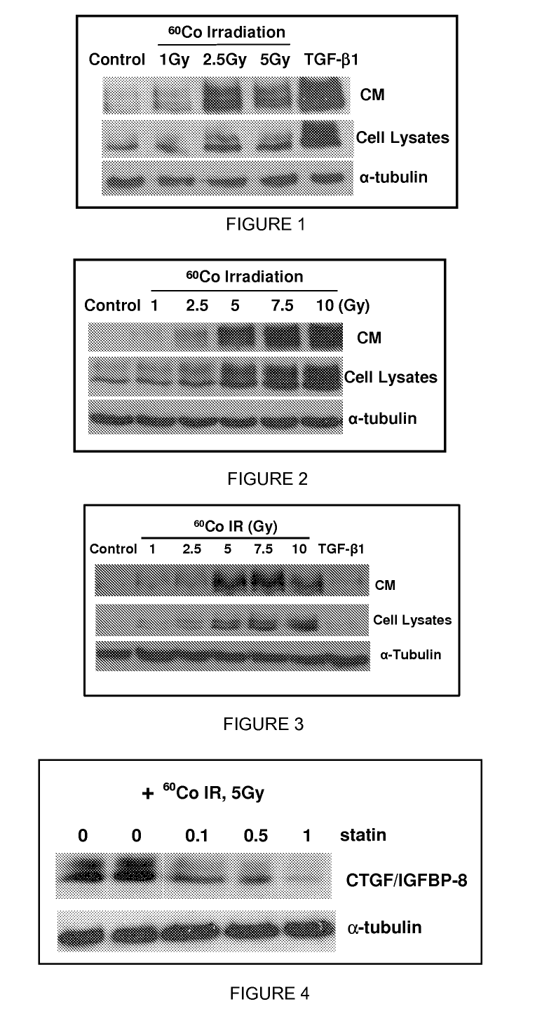

The present invention comprises an in vitro cell system for studying CTGF expression and for use in determining inhibitors of CTGF. For example, primary human lung fibroblasts and lung cancer cells can be used to study CTGF in RIF of the lung. As shown in FIG. 1, CTGF expression was increased by treatment with 5 ng/ml TGF-β1 for 4 days in HFL-1 human normal lung fibroblasts, as a positive control for CTGF induction in these cells. When normal lung fibroblasts and lung cancer cells were irradiated at doses ranging 1-5 Gy using a ⁶⁰Co-irradiator, there was an increase of CTGF expression in a dose-dependent manner at 3 day post-irradiation. Radiation exposure resulted in induction of CTGF not only in normal fibroblasts but also in cancer cells, as shown in FIGS. 2 and 3. An increase of CTGF expression after 1-10 Gy exposure was observed in lung cancer (FIG. 2) and prostate cancer cells (FIG. 3). Methods of the present invention comprise using an in vitro cell system to determine inhibitors of CTGF by comparing the amount of CTGF induced by radiation or TGF-β or other known inductive agents of CTGF, with the amount of inhibition of CTGF induction in the presence of known inhibitors and the compounds or compositions being tested for inhibition.

These and other examples herein indicate that radiation-induced CTGF in normal fibroblasts as well as cancer cells plays a role in RIF and may lead to enhanced expression of fibrotic factors such as fibronectin. Further studies demonstrated that radiation-induced CTGF expression may be suppressed in the in vitro cell systems by treatment with the statin compounds, HMG-CoA reductase inhibitors. As shown in FIG. 4, statin treatment inhibits radiation-induced CTGF expression in a concentration dependent manner.

Methods of the present invention comprise using an in vitro cell system to determine inhibitors of CTGF by comparing the amount of CTGF induced by radiation or TGF-β, or other known inductive agents of CTGF, with the amount of inhibition of CTGF induction in the presence of known inhibitors and the compounds or compositions being tested for inhibition.

These and other examples herein indicate that radiation-induced CTGF in normal fibroblasts plays a role in RIF and may lead to enhanced expression of fibrotic factors such as fibronectin. Further studies demonstrated that radiation-induced CTGF expression may be suppressed in the in vitro cell systems by treatment with statin compounds, HMG-CoA reductase inhibitors.

The present invention comprises methods for treatment or prevention of RIF in humans and animals by administering an effective amount of a compound or composition that prevents, inhibits or ameliorates formation of RIF. Methods for treatment or prevention of RIF comprise administration of compounds or compositions comprising HMG-CoA reductase inhibitors, referred to herein as statins, to a subject, human or animal, before undergoing, who is undergoing or has undergone exposure to ionizing radiation, such as radiation therapy. Methods for treatment or prevention of RIF comprise administration of compounds or compositions comprising inhibitors of CTGF, including but not limited to, statins, antibodies to CTGF, humanized or other antibodies that bind CTGF, antibody fragments or active sites that bind at least a portion of CTGF, interfering peptides, or interfering nucleic acids such as siRNA, or antisense RNA that interfere with or prevent the translation of CTGF RNAs or nucleic acids that interfere with or prevent the transcription of CTGF genes.

As referred to herein, statins or HMG-CoA reductase inhibitors, include, but are not limited to, compactin, lovastatin, simvastatin, pravastatin, fluvastatin, atorvastatin, mevastatin, rivastatin, cer(i)vastatin, pitavastatin, nisvastatin, itavastatin, rosuvastatin, berivastatin, dalvastatin, glenvastatin, RP 61969, SDZ-265859, BMS-180431, CP-83101, dihydromevinolin, L-669262, visastatin, or combinations thereof. HMG-CoA reductase inhibitors can have hypolipidemic properties due to their ability to inhibit HMG-CoA reductase, preventing the conversion of 3-hydroxy-3-methylglutary-1-CoA to mevalonate, which is the rate-limiting step in cholesterol synthesis. The chemical names are as follows: compactin (mevastatin, (2S)-2-methyl butanoic acid (1S,7S,8S,8aR)-1,2,3,7,8,8a-hexahydro-7-methyl-8-[2-[(2R,4R)-tetrahydro-4-hydroxy-6-oxo-2H-pyran-2-yl]ethyl]-1-naphthalenyl ester), lovastatin (2(S)-2-methyl-butanoic acid (1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthalenyl ester), simvastatin (2,2-dimethyl-butanoic acid (1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthalenyl ester), pravastatin((βR,δR, 1S,2S,6S,8S,8aR)-1,2,6,7,8,8a-hexahydro-β,β,6-trihydroxy-2-methyl-1-8-[(2S)-2-methyl-1-oxobutoxy]-1-naphthaleneheptanoic acid), fluvastatin ((3R,5S,6E)-rel-7-[3-(4-fluorophenyl)-1-(1-methylethyl)-1H-indol-2-yl]-3,-5-dihydroxy-6-heptenoic acid), rosuvastatin ((3R,5S,6E)-7-[4-(4-fluorophenyl)-6-(1-methylethyl)-2-[methyl(methylsulfonyl)amino]-5-pyrimidinyl]-3,5-dihydroxy-6-heptenoic acid), atovastatin((βR,δR)-2-(4-fluorophenyl)-β,δ-dihydroxy-5-(1-methylethyl)-3-phenyl-4-[(phenylamino)carbonyl]-1H-Pyrrole-1-heptanoic acid), pitavastatin ((3R,5 S,6E)-7-[2-cyclopropyl-4-(4-fluorophenyl)-3-quinolinyl]-3,5-dihydroxy-6-heptenoic acid), cervistatin ((3R,5S,6E)-7-[4-(4-fluorophenyl)-5-(methoxymethyl)-2,6-bis(1-methylethyl)-3-pyridinyl]-3,5-dihydroxy-6-heptenoic acid) berivastatin ((R*,S*-(E)-7-(4-(4-fluorophenyl)spiro(2H-1-benzopyran-2,1′-cyclopentan)-3-yl)-3,5-dihydroxy-ethyl ester), dalvastatin ((4R,6S)-rel-6-[(1E)-2-[2-(4-fluoro-3-methylphenyl)-4,4,6,6-tetramethyl-1-cyclohexen-1-yl]ethenyl]tetrahydro-4-hydroxy-2H-Pyran-2-one), glenvastatin ((4R,6S)-6-[(1E)-2-[4-(4-fluorophenyl)-2-(1-methylethyl)-6-phenyl-3-pyridinyl]ethenyl]tetrahydro-4-hydroxy-2H-Pyran-2-one), RP 61969 ([2S-[2a(E),4β]]-;4-(4-fluorophenyl)-2-(1-methylethyl)-3-[2-(tetrahydro-4-hydroxy-6-oxo-2H-pyran-2-yl)ethenyl]-1 (2H)-isoquinolinone), BMS-180431 ((3R,5S,6E)-rel-9,9-bis(4-fluorophenyl)-3,5-dihydroxy-8-(1-methyl-1H-tetrazol-5-yl)-6,8-Nonadienoic acid), CP-83101 ((3R,5 S,6E)-rel-3,5-dihydroxy-9,9-diphenyl-6,8-Nonadienoic acid methyl ester), dihydromevinolin ((2S)-2-methyl-butanoic acid (1S,3S,4aR,7S,8S,8aS)-1,2,3,4,4a,7,8,8a-octahydro-3,7-dimethyl-8-[2-[(2R,-4R)-tetrahydro-4-hydroxy-6-oxo-2H-pyran-2-yl]ethyl]-1-naphthalenyl ester), L-669262 (2,2-dimethyl-butanoic acid (1S,7R,8R,8aR)-1,2,6,7,8,8a-hexahydro-3,7-dimethyl-6-oxo-8-[2-[(2R,4R)-tetrahydro-4-hydroxy-6-oxo-2H-pyran-2-yl]ethyl]-1-naphthalenyl ester).

The specific structures of some of these preferred HMG-CoA reductase inhibitors are set forth below:

| | Chemical | |
|---|---|---|
| Name | Name | Structure |
| Compactin | (2S)-2-methyl-butanoic acid(1S,7S,8S,8aR)-1,2,3,7,8,8a-hexahydro-7-methyl-8-[2-[(2R,4R)-tetrahydro-4-hydroxy-6-oxo-2H-pyran-2-yl]ethyl]-1-naphthale-nylester | 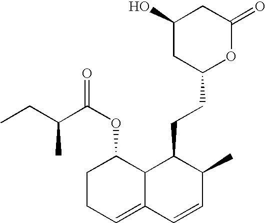 |
| Lovastatin | (2S)-2-methyl-butanoic acid(1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-na-phthalenylester | 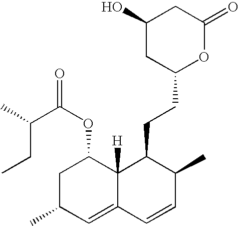 |
| Simvastatin | 2,2-dimethyl-butanoic acid(1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthalenylester | 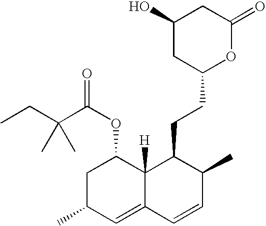 |
| Pravastatin | (βR,δR,1S,2S,6S,8S,8aR)-1,2,6,7,8,8a-hexahydro-β,δ,-6-trihydroxy-2-methyl-8-[(2S)-2-methyl-1-oxobutoxy]-1-naphthalene-heptanoic acid | 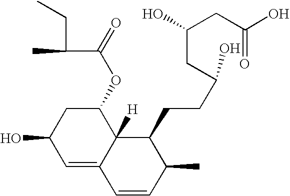 |
| Fluvastatin | (3R,5S,6E)-rel-7-[3-(4-fluorophenyl)-1-(1-methylethyl)-1H-indol-2-yl]-3,5--dihydroxy-6-heptenoic acid | 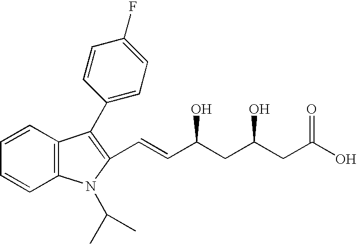 |
| Atorvastatin | (βR,δR)-2-(4-fluorophenyl)-β,δ-dihydroxy-5-(1-methylethyl)-3-phenyl-4(phenylamino)carbonyl]-1H-Pyrrole-1-heptanoic acid | 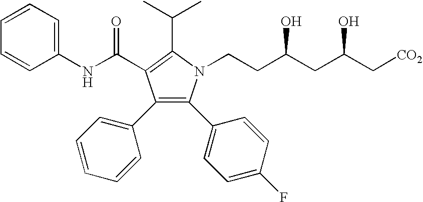 |
| Cerivastatin | (3R,5S,6E)-7-[4-(4-fluorophenyl)-5-(methoxymethyl)-2,6-bis(1-methylethyl)-3-pyridinyl]-3,5-dihydroxy-6-Heptenoicacid | 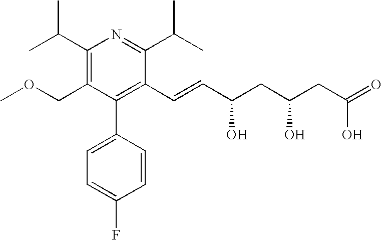 |
| Rosuvastatin | (3R,5S,6E)-7-[4-(4-fluorophenyl)-6-(1-methylethyl2-[methyl(methylsulfonyl-)amino]-5-pyrimidinyl]-3,5-dihydroxy-6-heptenoic acid | 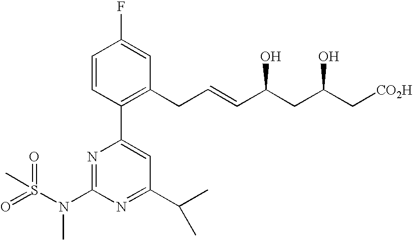 |

Other suitable HMG CoA reductase inhibitors are taught in U.S. Patent Application Pub. No. 2005/0239871, which is incorporated herein in its entirety.

The pharmaceutically acceptable salts and solvates, and prodrug forms of CTGF inhibitory compounds and compositions described herein can also be used in the methods of the present invention. Furthermore, derivatives of the compounds taught herein can also be used in the methods of the present invention. Derivatives include: derivatives of carboxylic acids (for example: carboxylic acid salts, esters, lactones, amides, hydroxamic acids, alcohols, esterified alcohols and alkylated alcohols (alkoxides)) and derivatives of alcohols (for example: esters, carbamates, lactones, carbonates, alkoxides, acetals, ketals, phosphates, and phosphate esters). Where a fluorine is found on one or more of the aromatic rings, any other halide can be used. Also, in lieu of hydrogen or alkyl groups, different alkyl groups can be used. For instance, instead of an —OH group, an —O-alkyl group could be used. Thus, various derivatives can easily be used in the present invention based on the guidance and knowledge presented herein, together with the knowledge that one skilled in the art has in this technical area. It is recognized that the compounds can contain one or more chiral centers. This invention contemplates all enantiomers, diastereomers, and mixtures thereof.

Methods of treating, preventing, or ameliorating RIF comprise administering an effective amount of a compound or composition that inhibits RIF to a human or animal. The compound or composition may be administered before the human or animal undergoes ionizing radiation therapy, during the period the human or animal is undergoing ionizing radiation therapy, or after the human or animal undergoes ionizing radiation therapy or combinations of these. For example, prior to radiation therapy, the patient is administered an effective amount of one or more statin compounds, such as in an oral dosage form. During radiation therapy, the patient is administered an effective amount of one or more statin compounds in an oral dosage form and optionally, also in a dosage form that supplies an amount of inhibitor compound to an effected area, such as by inhalation for thoracic or lung radiation, and after radiation therapy, for a continuous period of time, the patient is administered an oral dosage form of one or more statin compounds, optionally an inhalation dosage form, and optionally a topical dosage form comprising one or more statin compounds. The one or more statin compounds may be replaced by or used in addition to other inhibitory compositions including, but not limited to, antibodies to CTGF, humanized antibodies to CTGF, antibody fragments or active sites that bind to at least a portion of CTGF, interfering peptides, interfering nucleic acids, such as siRNA, or antisense RNA that interfere with or prevent the translation of CTGF RNAs or nucleic acids that interfere with or prevent the transcription of CTGF genes.

Methods and compositions of the present invention comprise administering compounds or compositions to inhibit CTGF expression or activity in a human or animal. CTGF, connective tissue growth factor (CTGF) is a cysteine-rich protein with a molecular weight of 36-38 kDa, and is known in the literature as CTGF, IGFBP-8 or CCN2, hereinafter referred to as CTGF. IGFBP-8 is insulin-like growth factor binding protein-8, and CCN2 is an alternate name for CTGF indicating it as a member of the CCN family, which stands for CTGF, CEF10/Cyr61, and Nov. Methods comprise administering an effective amount of a compound or composition to a human or animal to inhibit CTGF activity. Such compounds or compositions comprise statins, antibodies to CTGF, humanized antibodies to CTGF, antibody fragments or active sites for CTGF, interfering peptides, or interfering nucleic acids such as siRNA, or antisense RNA that interfere with or prevent the translation of CTGF RNAs or nucleic acids that interfere with or prevent the transcription of CTGF genes.

Methods and compositions of the present invention comprise administering an effective amount of a compound or composition to treat, ameliorate or prevent disorders resulting from CTGF activity in a human or animal. The compounds or compositions are inhibitors of CTGF expression or CTGF activity. A method of treating a disorder in a human or animal comprises administering an effective amount of a CTGF inhibiting compound or composition to a human or animal with a disorder due to CTGF activity. Such compounds or compositions comprise statins, antibodies to CTGF, humanized antibodies to CTGF, antibody fragments or active sites for CTGF, or interfering nucleic acids such as siRNA, or antisense RNA that interfere with or prevent the translation of CTGF RNAs or nucleic acids that interfere with or prevent the transcription of CTGF genes.

Methods and compositions of the present invention comprise treatment of humans or animals having neoplastic disease. Neoplastic disease may occur in any organ or tissue and may comprise cells that have uncontrollable growth, cells that may metastasize to other locations, and are commonly referred to as cancer, tumors or diffuse neoplastic tissue. A method of treating a neoplastic disease comprises administering an effective amount of a CTGF inhibiting compound or composition to a human or animal with neoplastic disease prior to, concurrently with or after radiation therapy for the neoplastic disease. Such compounds or compositions comprise statins, antibodies to CTGF, humanized antibodies to CTGF, antibody fragments or active sites for CTGF, interfering peptides, or interfering nucleic acids, such as siRNA, or antisense RNA that interfere with or prevent the translation of CTGF RNAs or nucleic acids that interfere with or prevent the transcription of CTGF genes.

In the methods of therapy of the present invention, and in the use of compositions according to the invention, a therapeutically effective amount of an RIF inhibitor or an effective amount of a CTGF inhibitor compound or composition can be administered to a subject requiring therapy. A “therapeutically effective amount” or “an effective amount” in the context of the present invention is considered to be any quantity of the one or more inhibitor compounds or compositions which, when administered to a subject prior to RIF formation but after exposure to ionizing radiation, suffering from RIF, or a CTGF related pathology, or a neoplastic disease, against which the inhibitor compound or compositions are effective, causes prevention, reduction, remission, or regression of RIF or the CTGF-related pathology.

The amount of the inhibitor compound or composition, such as a statin compound or derivative thereof, that can be used in the compositions or methods of the invention can be determined using assays for anti-fibrotic activity, the in vitro assays described herein, and by other methods, such as clinical trials, known to those skilled in the art. For example, therapeutically effective amounts of statins for use as anti-cholesterol agents are known and can be obtained from the appropriate supplier or, for example, the U.S. Food and Drug Association (www.fda.gov).

Antisense oligonucleotides are single-stranded nucleic acids which can specifically bind to a complementary nucleic acid sequence. By binding to an appropriate target sequence, an RNA-RNA, a DNA-DNA, or RNA-DNA duplex is formed. These nucleic acids are often termed “antisense” because they are complementary to the sense or coding strand of the gene. The oligonucleotide may also form a triple helix if bound to a DNA duplex. By binding to the target nucleic acid, oligonucleotides can inhibit the function of the target nucleic acid. This could, for example, be a result of blocking the transcription, processing, poly(A)addition, replication, translation, or promoting inhibitory mechanisms of CTGF, such as promoting RNA degradation.

Antisense oligonucleotides are prepared in the laboratory using standard laboratory protocols, as are known to those skilled in the art. The antisense molecules may then be supplied to a subject by, for example, injection, topical or mucosal application, or airway delivery. Antisense oligonucleotides may be 15 to 35 bases in length. However, it is appreciated that it may be desirable to use oligonucleotides with lengths outside this range, for example 10, 11, 12, 13, or 14 bases, or 36, 37, 38, 39 or 40 bases. The design of antisense molecules is routine and can readily be performed by the skilled person. By “antisense” it is intended to include all methods of RNA interference, which are regarded for the purposes of this invention as a type of antisense technology.

The present invention comprises use of antibodies for inhibition of CTGF, RIF and for use in the treatment and preventions methods disclosed herein. By “antibody” the term includes intact monoclonal and polyclonal antibody molecules as well as antibody fragments (such as, for example, Fab and F(ab′)2 fragments). Fab and F(ab′)2 fragments lack the Fc fragment of intact antibody, clear more rapidly from the circulation, and may have less non-specific tissue binding of an intact antibody. Such antibodies may be humanized or not, and may have functional groups or tags associated with them for monitoring functions or additional activities.

Compositions of the present invention may be formulated according to protocols well known in the art. Suitable formulations may be determined based on the preferred route by which the medicament is to be administered. Compositions of the invention may be prepared in forms suitable for administration by oral dosage forms known in the pharmaceutical arts, including, but not limited to, tablets, capsules, oral liquid formulas, quick dissolve tablets, buccal and other mucosal dosage formulas, inhalation, topical administration, ophthalmic administration, by injection, or by implantation. For example, in methods of treatment of RIF, inhibition of CTGF, or neoplastic disease in the lung, the inhibitor compound or composition such as a statin or statin derivative may be prepared as an aerosol for delivery intranasally or by inhalation to the lungs, or may be provided in an oral dosage formulation.

Compositions, such as statins or statin derivatives, administered intranasally or by inhalation can be delivered in the form of a dry powder inhaler or an aerosol spray presentation from a pressurized container, pump, spray or nebulizer with the use of a suitable propellant, carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are provided so that each metered dose contains a suitable quantity of an inhibitor compound or composition, such as statins or statin derivatives, for delivery to the subject. It will be appreciated that the overall daily dose with an aerosol will vary from patient to patient, and may be administered in a single dose or, more usually, in divided doses throughout the day. Nanoparticulated compounds or compositions, such as statins or statin derivatives, may be prepared using techniques known in the art.

Methods of treatment or prevention of RIF, inhibition of CTGF, or neoplastic disease in the eye may require that the inhibitor compound or composition, such as a statin or statin derivative, may be prepared as a liquid formulation to the eye. For ophthalmic use, the inhibitor compound or compositions, such as statins or statin derivatives, may be formulated as micronized suspensions in isotonic, pH adjusted, sterile saline, or, preferably, as solutions in isotonic, pH adjusted, sterile saline, optionally in combination with a preservative such as a benzylalkonium chloride.

Methods of treatment or prevention of RIF, inhibition of CTGF, or neoplastic disease in the skin or integumentary system may require that the inhibitor compound or composition, such as a statin or statin derivative, may be prepared for topical administration directly to the skin. For topical application to the skin, the inhibitor compound or compositions, such as statins or statin derivatives, can be formulated as a suitable ointment containing one or more active compounds or compositions suspended or dissolved in, for example, a mixture with one or more of the following: mineral oil, liquid petrolatum, white petrolatum, propylene glycol, polyoxyethylene polyoxypropylene compound, emulsifying wax, and water. Alternatively, formulations may be a lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol, and water.

Compositions of the present invention comprise formulations known for administration of therapeutic agents. The compositions may be in the form of a powder, tablet, capsule, liquid, ointment, cream, gel, hydrogel, aerosol, spray, micelle, transdermal patch, liposome or any other suitable form that may be administered to a person or animal

Liquid vehicles are used in preparing solutions, suspensions, emulsions, syrups, elixirs, and pressurized compositions. The liquid vehicle can contain suitable pharmaceutical additives such as solubilizers, emulsifiers, buffers, preservatives, sweeteners, flavoring agents, suspending agents, thickening agents, colors, viscosity regulators, stabilizers, or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester, such as ethyl oleate and isopropyl myristate. Sterile liquid vehicles are useful in sterile liquid form compositions for parenteral administration. The liquid vehicle for pressurized compositions can be halogenated hydrocarbons or other pharmaceutically acceptable propellant.

Liquid pharmaceutical compositions, which are sterile solutions or suspensions can be utilized by, for example, intramuscular, intrathecal, epidural, intraperitoneal, or subcutaneous injection. Sterile solutions can also be administered intravenously. The inhibitory compounds or compositions may be prepared as a sterile solid composition, which may be dissolved or suspended at the time of administration using sterile water, saline, or other appropriate sterile injectable medium. Vehicles are intended to include necessary and inert binders, suspending agents, lubricants, flavorants, sweeteners, preservatives, dyes, and coatings.

Optimal dosages to be administered for the treatment or prevention of RIF, for inhibition of CTGF, or treatment of neoplastic disease may be determined by those skilled in the art, and will vary with the particular disease, patient and treatment or prevention protocol in use, the strength of the preparation, the mode of administration, and the advancement of the disease condition that is to be treated. Additional factors depending on the particular subject being treated will result in a need to adjust dosages, including subject age, weight, gender, diet, and time of administration.

Known procedures, such as those conventionally employed by the pharmaceutical industry (e.g. in vivo experimentation, clinical trials etc), may be used to establish specific formulations of compositions and precise therapeutic regimes (such as daily doses of the inhibitory compounds and compositions, and the frequency of administration). Daily doses may be given as a single administration (e.g. a daily tablet for oral consumption or as a single daily injection). Alternatively, the inhibitory compounds and compositions used may require administration two or more times during a day, dependent on pharmacological, toxicological or efficacy studies.

Administration of an effective dose of an inhibitory compound or composition may occur before onset of radiation therapy, during radiation therapy, or after radiation therapy to prevent or treat CTGF related disease, RIF or in treatment of neoplastic disease. The doses may be administered daily, more than one time a day, weekly, monthly or over one or more years to treat or prevent RIF, CTGF related pathologies and in treating neoplastic disease. An effective dose may comprise from 0.02 μg to 200 mg/kg patient of an HMG-CoA reductase inhibitor compound, or from 0.001 μg to 1,000 mg/kg patient of antibodies that bind to at least a portion of CTGF, antisense molecules comprising CTGF sequences, humanized antibodies that bind CTGF, antibody fragments or active sites that bind at least a portion of CTGF, interfering peptides, or interfering nucleic acids such as siRNA, or antisense RNA or nucleic acids that interfere with or prevent the transcription of CTGF genes.

In general, the present invention comprises methods comprising administering or providing compounds or compositions that inhibit CTGF to humans or animals in need thereof, or to in vitro cell systems to determine inhibition of CTGF. As used herein, inhibiting CTGF means inhibiting, lessening or stopping one or more activities of CTGF, or interfering with effective action of CTGF in cellular pathways in which CTGF is active, for example as a signaling factor or cytokine. Inhibiting CTGF leads to a reduction, amelioration or lessening of cellular and tissue pathologies related to actions by CTGF in cells. For example, inhibiting CTGF results in treatment, reduction, amelioration, lessening, or prevention of radiation-induced fibrosis.

Radiation-induced fibrosis is associated with exposure by humans or animals to ionizing radiation. The ionizing radiation may result from radiation therapy for neoplastic disease, or may result from exposure to ionizing radiation from other sources such as radionuclide contamination, nuclear weapons, mining activities, accidental exposures from nuclear power generation, industrial hazards, or other exposures to ionizing radiation. Ionizing radiation is highly-energetic particles or waves that can detach (ionize) at least one electron from an atom or molecule. Ionizing ability depends on the energy of individual particles or waves, and not on their number. A large flood of particles or waves will not, in the most common situations, cause ionization if the individual particles or waves are not by themselves ionizing. Examples of ionizing radiation are energetic beta particles, neutrons, and alpha particles. The ability of light waves (photons) to ionize an atom or molecule varies across the electromagnetic spectrum. X-rays and gamma rays can ionize almost any molecule or atom; far ultraviolet light can ionize many atoms and molecules; near ultraviolet and visible light are ionizing to very few molecules; microwaves and radio waves are non-ionizing radiation. Visible light is so ubiquitous that molecules that are ionized by it often react nearly spontaneously unless protected by materials that block the visible spectrum.

Electrons, x rays, gamma rays or atomic ions may be used in radiation therapy to treat neoplastic disease, including malignant tumors (cancer). Medical procedures, such as diagnostic X-rays, nuclear medicine, and radiation therapy are by far the most significant source of human-made radiation exposure to the general public. Some of the major radionuclides used are I¹³¹, Tc⁹⁹, Co⁶⁰, Ir¹⁹², and Cs¹³⁷. Humans and animals are exposed to radiation from consumer products, such as tobacco (Po²¹⁰), building materials, combustible fuels (gas, coal, etc.), ophthalmic glass, televisions, luminous watches and dials (H³), airport X-ray systems, smoke detectors (Amercium (Am)), road construction materials, electron tubes, fluorescent lamp starters, and lantern mantles (thorium (Th)). Occupationally exposed individuals are exposed according to the sources with which they work. Some of the radionuclides of concern include Co⁶⁰, Cs¹³⁷, Am²⁴¹, and I¹³¹. Examples of industries where occupational exposure is a concern include airline crew, industrial radiography, nuclear medicine and medical radiology departments (including nuclear oncology), nuclear power plants and research laboratories.

A method of the present invention for treating or preventing radiation-induced fibrosis comprises administering to a human or animal, an effective amount of a composition that inhibits CTGF. The composition that inhibits CTGF may be administered to the human or animal prior to exposure of the human or animal to ionizing radiation, concurrently with exposure of the human or animal to ionizing radiation, after the human or animal is exposed to ionizing radiation, or a combination of two or more of these. Administration concurrently with radiation may include administration of one or more compositions that inhibit CTGF on the same day as ionizing radiation is provided, administration every day during the time period in which a course of ionizing radiation exposures are provided, or on one or more days during the time period in which a course of ionizing radiation exposures are provided. Typically ionizing radiation is provided for treatment of neoplastic disease or cancer over a time period of days, weeks or months, or until a particular amount of radiation exposure has been reached by the target area of the human or animal body. As used herein, neoplastic disease means the occurrence of abnormal new growth of tissue that grows by cellular proliferation more rapidly than normal, continues to grow after the stimuli that initiated the new growth cease, may show partial or complete lack of structural organization and functional coordination with the normal tissue, and may forms a distinct mass of tissue which may be either benign or malignant. In common understanding, treatment of neoplastic disease is treatment of cancer or uncontrolled growth of cells, whether in the original location of the cells or in metastases.

Compositions comprising inhibitors of CTGF comprise at least one HMG-CoA reductase inhibitor compound or combinations of HMG-CoA reductase inhibitor compounds. HMG-CoA reductase inhibitor compounds include compactin, lovastatin, simvastatin, pravastatin, fluvastatin, atorvastatin, mevastatin, rivastatin, cer(i)vastatin, pitavastatin, nisvastatin, itavastatin, rosuvastatin, berivastatin, dalvastatin, glenvastatin, dihydromevinolin and visastatin. An HMG-CoA reductase inhibitor compound is simvastatin. Compositions comprising one or more statin (HMG-CoA reductase inhibitor compound) compounds may further comprise CTGF inhibitors comprising antibodies that bind to at least a portion of CTGF, antisense molecules comprising CTGF sequences, humanized antibodies that bind CTGF, antibody fragments or active sites that bind at least a portion of CTGF, interfering peptides, or interfering nucleic acids such as siRNA, or antisense RNA, or nucleic acids that interfere with or prevent the transcription of CTGF genes.

A method of the present invention comprises treating or preventing a pathology related to CTGF activity, comprising, administering to a human or animal an effective amount of a composition that inhibits CTGF activity. A pathology related to CTGF activity is radiation-induced fibrosis. Compositions that inhibit CTGF comprise at least one HMG-CoA reductase inhibitor compound, a combination of HMG-CoA reductase inhibitor compounds, antibodies that bind to at least a portion of CTGF, antisense molecules comprising CTGF sequences, humanized antibodies that bind CTGF, antibody fragments or active sites that bind at least a portion of CTGF, interfering peptides, or interfering nucleic acids such as siRNA, antisense RNA, nucleic acids that interfere with or prevent the transcription of CTGF genes, of combinations thereof.

An aspect of neoplastic disease treatment that includes radiation therapy using ionizing radiation is sequellae due to radiation-induced fibrosis. Thus a treatment of neoplastic disease comprising ionizing radiation comprises treatment or prevention of radiation-induced fibrosis. A method for treating or preventing radiation-induced fibrosis comprises administering compositions comprising inhibitors of CTGF. A method for treating neoplastic disease comprises administering an effective amount of a composition that inhibits CTGF to a human or animal, and irradiating the human or animal with an effective amount of ionizing radiation to affect the neoplastic disease. The CTGF inhibiting composition may be administered at any time, prior to, during, and after exposure to ionizing radiation. Compositions that inhibit CTGF comprise at least one HMG-CoA reductase inhibitor compound, a combination of HMG-CoA reductase inhibitor compounds, antibodies that bind to at least a portion of CTGF, antisense molecules comprising CTGF sequences, humanized antibodies that bind CTGF, antibody fragments or active sites that bind at least a portion of CTGF, interfering peptides, or interfering nucleic acids such as siRNA, antisense RNA, nucleic acids that interfere with or prevent the transcription of CTGF genes, of combinations thereof.

Methods of the present invention comprise use of an in vitro cell system to determine the CTGF inhibitory activity of compounds. A method comprises using cells in which CTGF can be induced, such as by cellular factors such as TGF-β or by ionizing radiation, and dividing the cells in several experimental groups. In one group of cells, the cells are induced to express or produce CTGF. In another group of cells, the cells are induced to express or produce CTGF and the cells are treated with a known CTGF inhibitor such as by adding at least one HMG-CoA reductase inhibitor compound, a combination of HMG-CoA reductase inhibitor compounds, antibodies that bind to at least a portion of CTGF, antisense molecules comprising CTGF sequences, humanized antibodies that bind CTGF, antibody fragments or active sites that bind at least a portion of CTGF, interfering peptides, or interfering nucleic acids such as siRNA, antisense RNA, nucleic acids that interfere with or prevent the transcription of CTGF genes, of combinations thereof, before, after or concurrently with inducing the cells. In a third set of cells, CTGF is induced and the compound under investigation for inhibition of CTGF activity is added before, after or concurrently with inducing the cells. A comparison of the level of CTGF produced or expressed in each of the sets of cells leads to the determination of whether the compound under investigation is an inhibitor of CTGF.

It must be noted that, as used in this specification and the appended claims, the singular forms “a”, “an”, and “the” include plural referents unless the context clearly dictates otherwise.

All patents, patent applications and references included herein are specifically incorporated by reference in their entireties.

It should be understood, of course, that the foregoing relates only to exemplary embodiments of the present invention and that numerous modifications or alterations may be made therein without departing from the spirit and the scope of the invention as set forth in this disclosure.

Although the exemplary embodiments of the present invention are provided herein, the present invention is not limited to these embodiments. There are numerous modifications or alterations that may suggest themselves to those skilled in the art.

The present invention is further illustrated by way of the examples contained herein, which are provided for clarity of understanding. The exemplary embodiments should not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1

### Materials and Methods

Cell Culture and Treatment. Normal human fetal lung fibroblasts (HFL-1) were purchased from the American Type Culture Collection (ATCC Number: CCL-153) and cultured in Ham's F12K medium (American Type Culture Collection) supplemented with 2 mM L-glutamine adjusted to contain 1.5 g/L sodium bicarbonate and 10% fetal bovine serum. Cells were maintained in humidified 5% CO₂ at 37° C. Human dermal fibroblasts (HDF), derived from the dermis of normal human adult skin, were purchased from Invitrogen, Inc. and cultured in Medium-106 (Invitrogen, Inc., Catalogue No. M-106-500) containing LSGS Kit (Invitrogen, Inc., Catalogue No. S-003-K) prior to use. After reaching 75-80% confluence, the medium was changed to serum free medium (SFM) for irradiation with different dose 1Gy, 2.5Gy, 5Gy, 7.5Gy and 10 Gy by using ¹³⁷Se. Alternatively, cells were treated with 5 ng/ml TGF-β1 (Sigma-Aldrich, Inc., Catalogue No. T7039) to stimulate CTGF production. The cells were then kept for 3 days in humidified 5% CO₂ at 37° C. and subjected to Western immunoblot.¹ ¹What are the culture conditions (e.g., media) for M12 cells used in FIG. 3?

For statin treatment experiments, cells were grown overnight in 35 mm plates (6-well plates were also used in some studies) to form a 75-80% confluent monolayer. The cells were then washed with PBS, and the media was changed to SFM (800 μL). Cells were treated with the indicated amount of simvastatin (Sigma-Aldrich, Inc., Catalogue No. S6196), mevastain (Sigma-Aldrich, Inc., Catalogue No. M2537), mevinolin (Sigma-Aldrich, Inc., Catalogue No. M2147), pravastatin sodium (Sigma-Aldrich, Inc., Catalogue No. P4498), or SR 12813 (Sigma-Aldrich, Inc., Catalogue No. S4194) for six (6) hours before subjected to 5 Gy irradiation and were incubated in humidified 5% CO₂ at 37° C. for three (3) days in serum free medium post irradiation.

Harvesting Culture Media and Cell Lystates. For analysis of extracellular CTGF, fibronectin, and α-tubulin, cell culture media was collected, centrifuged to remove cellular debris, aliquoted, and frozen at −70° C. until ready for use. For analysis of intracellular CTGF, fibronectin, and α-tubulin, cell lysates were harvested using 200 μL of HBSST lysis buffer (HBSS containing 1 mM MgSO₄, 1 mM CaCl₂, 4 mM NaHCO₃, 0.5% Triton X-100, protease inhibitor cocktail). Cellular debris was removed by centrifugation.

RT-PCR analysis of CTGF, COL-IV, FN and hβ₂M. Total RNA was extracted from cells treated with radiation, or TGF-β1, or irradiation plus simvastatin, or TGF-β1 plus simvastatin, or a no treatment control following six (6) hour and 20 hour incubation using the TRIZOL (Invitrogen, Inc.) extraction technique, as recommended by the manufacturer. One microgram of purified total RNA was used for RT-PCR analysis using the ThermoScript RT-PCR System (Invitrogen, Inc.) according to the protocol of the manufacturer. The sequences of the forward and reverse primers are as follows: CTGF forward primer (SEQ ID NO 1) 5′-CTGGTCCAGACCACAGAGTG-3′, CTGF reverse primer (SEQ ID NO 2) 5′-CGGTATGTCTTCATGCTGGT-3′; COL-IV forward primer (SEQ ID NO 3) 5′-AGCAAGGCAACAGAGGACTT-3′, COL-IV reverse primer (SEQ ID NO 4) 5′-GATCTGGGTGGAAGGTGACT-3′; FN forward primer (SEQ ID NO 5) 5′-GACTGGAGCTGGAGACATGA-3′, FN reverse primer (SEQ ID NO 6) 5′-GTGATGATGGTGGACTGCTC-3′; hβ₂M forward primer (SEQ ID NO 7) 5′-GTGCTCGCGCTCTCTCT-3′; hβ₂M reverse primer (SEQ ID NO 8) 5′-CGGCAGGCATACTCATCTTT-3′. The CTGF PCR product is 242 bp in length, the COL-IV PCR product is 138 bp, the FN PCR product is 203 bp, and hβ₂M PCR product is 278 bp. For PCR amplification, 25 cycles were performed for the amplification of CTGF, COL-IV, FN and hβ₂M (denaturation: 94° C., 45 sec; annealing: 55° C., 45 sec; polymerization: 72° C., 1 min). Amplified products were separated by electrophoresis on a 1.0% agarose gel, and DNA was visualized by ethidium bromide staining.

Western blot analysis of HFL-1 whole cell proteins. The primary antibodies that selectively recognized CTGF (sc-14939) and fibronectin (sc-9068) were purchased from Santa Cruz Biotechnology, Inc., and primary antibodies that selectively recognized α-tubulin (T 9026) were purchased from Sigma. Primary antibodies for CTGF and fibronectin were diluted at 1:600 and were incubated at 4° C. overnight with shaking. Primary antibodies for α-tubulin were diluted at 1:4,000 and were incubated at 4° C. overnight with shaking. Corresponding secondary antibodies were diluted at 1:6,000 and were incubated at room temperature for 1 hour with shaking. After washing the blot, the antibodies were detected by the enhanced chemiluminescence method (PerkinElmer Life Sciences Inc.).

### Example 2

### Induction of CTGF, Fibronectin, and Collagen Type IV with TGF-β1

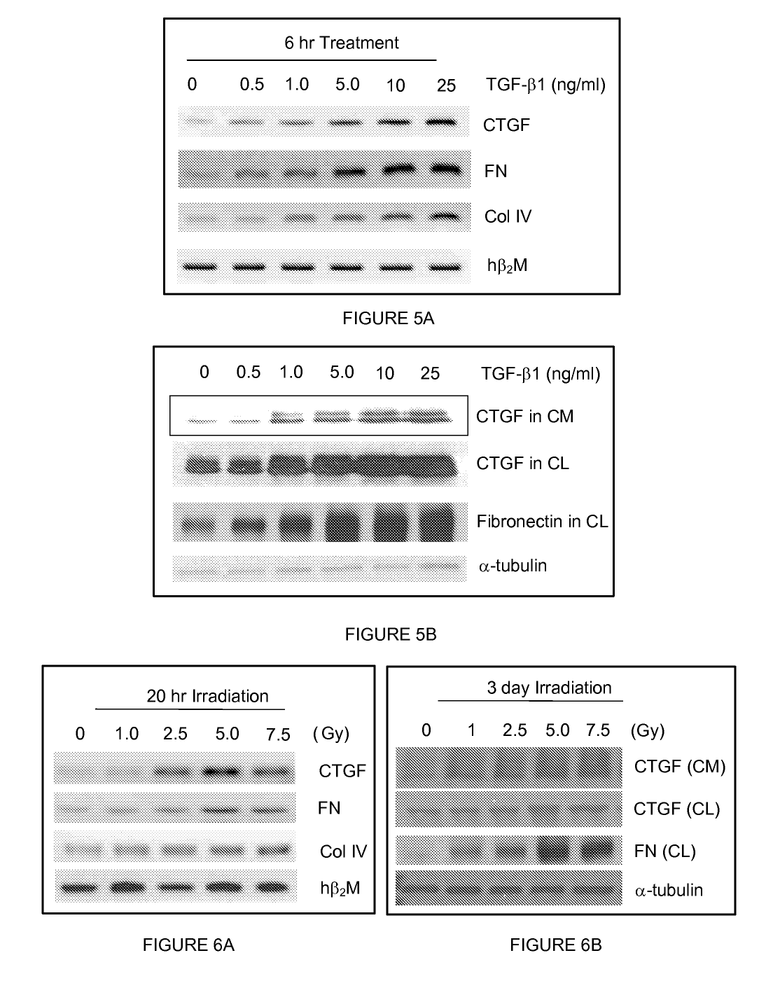

Normal human fetal lung fibroblasts (HFL-1) were treated with varying concentrations of TGF-β1, ranging from 0 ng/ml to 25 ng/ml. In order to detect expression of CTGF and fibrotic proteins mRNAs, RNA was extracted from HFL-1 cells treated with TGF-β1, and RT-PCR was performed to specifically detect expression of CTGF, fibronectin (FN), collagen type IV (Col IV), and hβ₂M mRNA. FIG. 5A is an agarose gel of RT-PCR products detected in HFL-1 cells following a six (6) hour treatment with TGF-β1. As shown in FIG. 5A, expression of CTGF, FN, and Col IV mRNA increased as the concentration of TGF-β1 increased. hβ₂M mRNA was used as an internal control for the RT-PCR process.

Protein expression in HFL-1 cells treated with TGF-β1 was also determined. Three days following treatment with the indicated amount of TGF-β1, HFL-1 cells were harvested, and cell lysates were separated by SDS-PAGE. The separated proteins were then transferred to nitrocellulose, and Western immunoblotting was performed with antibodies specific for CTGF, fibronectin, and α-tubulin. FIG. 5B is a Western blot of HFL-1 cells treated with TGF-β1. Similar to the results of mRNA expression in FIG. 5A, the amount of CTGF in both the culture medium (CM) and the cell lysate (CL) increased as the amount of TGF-β1 increased. The same increase in protein expression was also observed for fibronectin. As a control for protein loading, α-tubulin expression in HFL-1 cells was also determined.

### Example 3

### Induction of CTGF, Fibronectin, and Collagen Type IV with Radiation

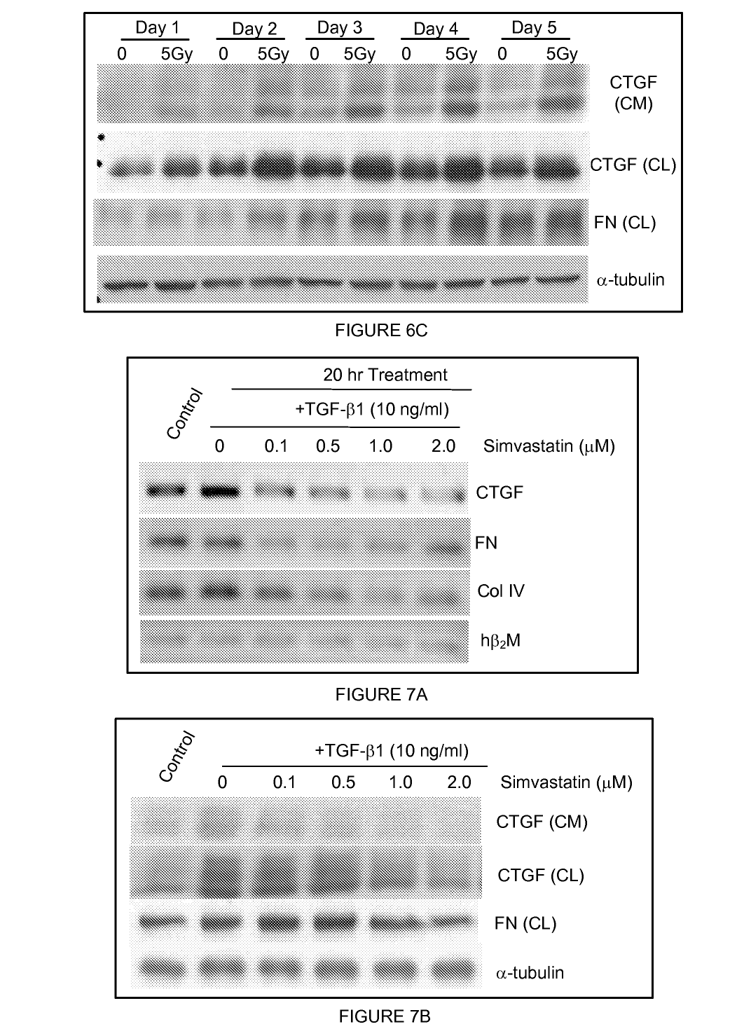

In an effort to determine the mechanism of radiation-induced fibrosis, HFL-1 cells were exposed to increasing amounts of radiation, ranging from 0 to 7.5 Grays (Gy). To detect mRNA expression of CTGF and fibrotic proteins mRNAs, RNA was extracted from irradiated HFL-1 cells, and RT-PCR was performed to specifically detect expression of CTGF, fibronectin (FN), collagen type IV (Col IV), and hβ₂M mRNA. FIG. 6A is an agarose gel of an RT-PCR analysis of mRNA expression in HFL-1 cells collected 20 hours following exposure to radiation. As shown in FIG. 6A, the expression of CTGF and fibrotic protein mRNA increased as the amount of radiation increased.

Protein expression in HFL-1 cells exposed to radiation was also determined. Three days following exposure to radiation, HFL-1 cells were harvested, and cell lysates were separated by SDS-PAGE. The separated proteins were then transferred to nitrocellulose, and Western immunoblotting was performed with antibodies specific for CTGF, fibronectin, and α-tubulin. FIG. 6B is a Western blot of HFL-1 cells exposed to radiation. Similar to the results of mRNA expression in FIG. 6A, the amount of CTGF in both the culture medium (CM) and the cell lysate (CL) increased as the amount of radiation increased. The same increase in protein expression was also observed for fibronectin. As a control for protein loading, α-tubulin expression in HFL-1 cells was also determined.

A time course study of CTGF and fibronectin expression in response to irradiation was also conducted. HFL-1 cells were either exposed to no radiation (control) or 5 Gy of radiation over a five day period. On each day, HFL-1 cells were harvested, and cell lysates were separated by SDS-PAGE. The separated proteins were then transferred to nitrocellulose, and Western immunoblotting was performed with antibodies specific for CTGF, fibronectin, and α-tubulin, as shown in FIG. 6C. FIG. 6C illustrates a time-dependent increase of CTGF and FN protein expression following exposure to 5 Gy radiation. During the time course of the experiment, the amount of CTGF in both the culture medium (CM) and the cell lysate (CL) increased as the days exposed to radiation increased. The same temporal increase in protein expression was also observed for fibronectin. As a control for protein loading, α-tubulin expression in HFL-1 cells was also determined.

### Example 4

### Inhibitory Effect of Simvastatin on TGF-β1-Induction of CTGF, Fibronectin, and Collagen Type IV

The ability of statins to inhibit TGF-β1-mediated induction of CTGF, fibronectin, and collagen type IV was assessed in HFL-1 cells. In this experiment, cells were treated with the indicated amount of simvastatin in the presence of 10 ng/ml of TGF-β1 for 20 hours. In order to determine if simvastatin would affect expression of CTGF and fibrotic proteins mRNAs, RNA was extracted from HFL-1 cells treated with both TGF-β1 and simvastatin, and RT-PCR was performed to specifically detect expression of CTGF, fibronectin (FN), collagen type IV (Col IV), and hβ₂M mRNA. FIG. 7A is an agarose gel of an RT-PCR analysis of mRNA expression in HFL-1 cells treated with TGF-β1 and treated with simvastatin. In the presence of TGF-β1 but the absence of simvastatin, expression of CTGF, FN, and Col IV mRNA increased relative to control cells, similar to that observed in FIG. 5A. Upon the addition of 0.1 μM simvastatin, a reduction in CTGF, FN, and Col IV mRNA expression was observed. As the concentration of simvastatin increased, the reduction in CTGF, FN, and Col IV mRNA expression became more pronounced. To control for the RT-PCR process, expression of hβ₂M mRNA was used as a control.

Protein expression in HFL-1 cells treated with both TGF-β1 and simvastatin was also determined. Three days following treatment with both simvastatin and TGF-β1, HFL-1 cells were harvested, and cell lysates were separated by SDS-PAGE. The separated proteins were then transferred to nitrocellulose, and Western immunoblotting was performed with antibodies specific for CTGF, fibronectin, and α-tubulin. FIG. 7B is a Western blot of HFL-1 cells treated with both TGF-β1 and simvastatin. Similar to the results of mRNA expression in FIG. 7A, the amount of CTGF detected in both the culture medium (CM) and the cell lysate (CL) decreased as the amount of simvastatin increased. A decrease in protein expression or fibronectin was also observed. As a control for protein loading, α-tubulin expression in HFL-1 cells was also determined.

### Example 5

### Inhibitory Effect of Simvastatin on Radiation-Induction of CTGF, Fibronectin, and Collagen Type IV

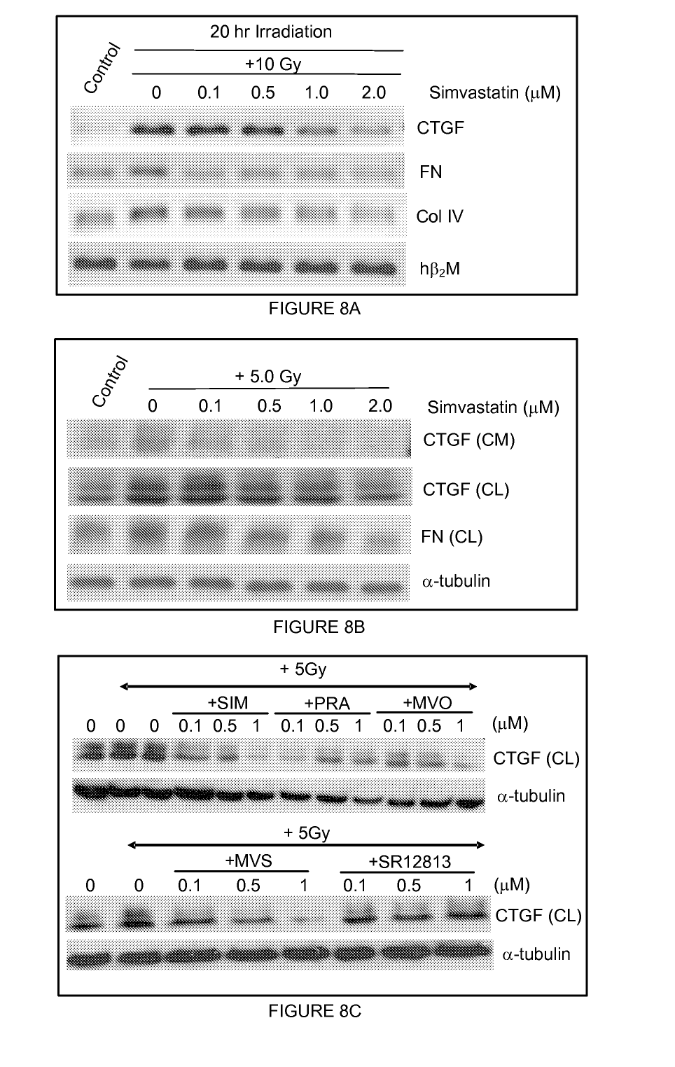

In order to determine the effects of simvastatin on irradiated cells, the expression of CTGF, fibronectin, and collagen type IV mRNA was assessed in irradiated HFL-1 cells. As shown in FIG. 8A, cells were pretreated with the indicated amount of simvastatin for six (6) hours, were then subjected to 10 Gy of radiation, and were then incubated for 20 hours. To determine if simvastatin affected expression of CTGF mRNA and fibrotic proteins mRNAs, RNA was extracted from HFL-1, and RT-PCR was performed to specifically detect expression of CTGF, fibronectin (FN), collagen type IV (Col IV), and hβ₂M mRNA. FIG. 8A is an agarose gel of an RT-PCR analysis of mRNA expression in HFL-1 cells exposed to radiation and treated with simvastatin. In comparison to control cells, irradiation of cells receiving no simvastatin resulted in expression of both CTGF mRNA as well as FN and Col-IV mRNA (See FIG. 8A, lane 2). As the amount of simvastatin increased, a decrease in the expression of CTGF, FN, and Col-IV mRNA was observed.

Protein expression in irradiated HFL-1 cells treated with simvastatin was also determined. In the experiment shown in FIG. 8B, cells were pretreated with the indicated amount of simvastatin for six (6) hours, were subjected to 5 Gy of radiation, and were then incubated for three (3) days. Cells were harvested, and cell lysates were separated by SDS-PAGE. The separated proteins were then transferred to nitrocellulose, and Western immunoblotting was performed with antibodies specific for CTGF, fibronectin, and α-tubulin. FIG. 8B is a Western blot of HFL-1 cells exposed to radiation and treated with simvastatin. Similar to the results for mRNA expression in FIG. 8A, the amount of CTGF detected in both the culture medium (CM) and the cell lysate (CL) decreased as the amount of simvastatin increased. A decrease in protein expression or fibronectin was also observed. As a control for protein loading, α-tubulin expression in HFL-1 cells was also determined.

In order to determine if the inhibitory effect of simvastatin on radiation-induced CTGF expression was applicable to other HMG-CoA inhibitors, HFL-1 cells were pretreated with the indicated amount of HMG-CoA inhibitors (SIM, simvastatin; PRA, pravastatin; MVO, mevinolin; MVS, mevastatin; and SR12813) for six (6) hours, were subjected to 5 Gy of radiation, and were then incubated for three (3) days. Cells were harvested, and cell lysates were separated by SDS-PAGE. The separated proteins were then transferred to nitrocellulose, and Western immunoblotting was performed with antibodies specific for CTGF, fibronectin, and α-tubulin. FIG. 8C is a Western blot of HFL-1 cells exposed to radiation and treated with HMG-CoA inhibitors. As shown in FIG. 8C, the amount of CTGF detected in the cell lysate (CL) decreased as the amount of HMG-CoA inhibitor increased; however the inhibitory effects for SR12813 were reduced relative to the inhibitory effects of simvastatin, pravastatin, lovastatin, mevinolin, and mevastatin.

### Example 6

### Induction of CTGF, Fibronectin, and Collagen Type IV with TGF-β1

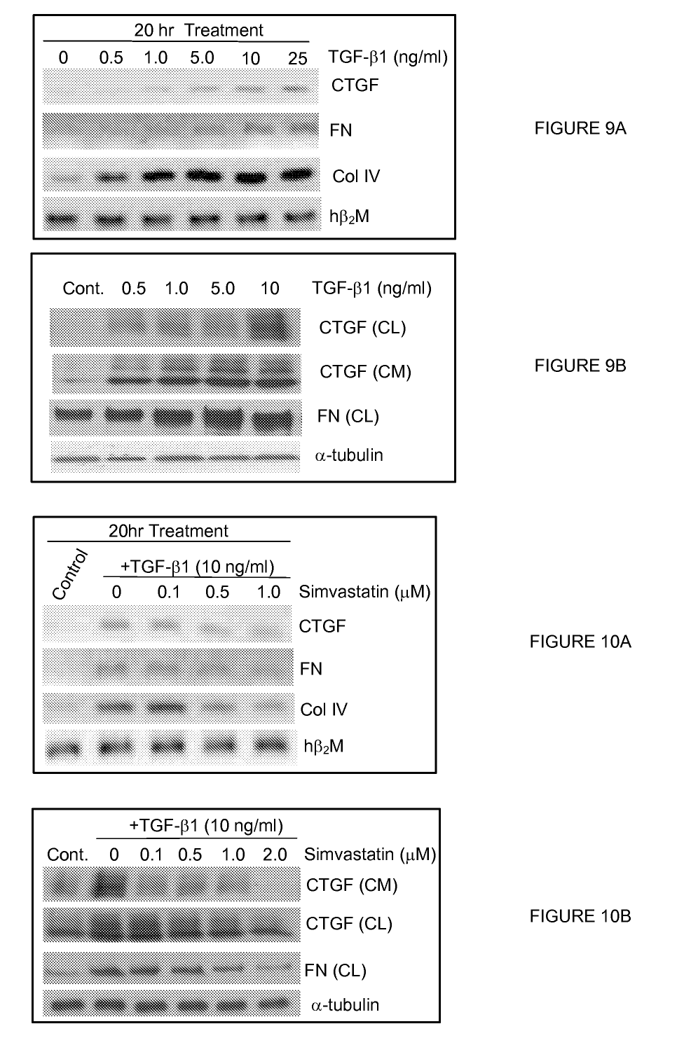

Human dermal fibroblasts (HDF) were treated with varying concentrations of TGF-β1, ranging from 0 ng/ml to 25 ng/ml, for twenty hours. In order to detect expression of CTGF and fibrotic proteins mRNAs, RNA was extracted from HDF cells treated with TGF-β1, and RT-PCR was performed to specifically detect expression of CTGF, fibronectin (FN), collagen type IV (Col IV), and hβ₂M mRNA. FIG. 9A is an agarose gel of RT-PCR products detected in HDF cells following a six (6) hour treatment with TGF-β1. As shown in FIG. 9A, expression of CTGF, FN, and Col IV mRNA increased as the concentration of TGF-β1 increased. hβ₂M mRNA was used as an internal control for RT-PCR.

Protein expression in HDF cells treated with TGF-β1 was also determined. Three days following treatment with TGF-β1, HDF cells were harvested, and cell lysates were separated by SDS-PAGE. The separated proteins were then transferred to nitrocellulose, and Western immunoblotting was performed with antibodies specific for CTGF, fibronectin, and α-tubulin. FIG. 9B is a Western blot of HDF cells treated with TGF-β1. Similar to the results for mRNA expression in FIG. 9A, the amount of CTGF in both the culture medium (CM) and the cell lysate (CL) increased as the amount of TGF-β1 increased. The same increase in protein expression was also observed for fibronectin. As a control for protein loading, α-tubulin expression in HFL-1 cells was also determined.

### Example 7

### Inhibitory Effect of Simvastatin on TGF-β1-Induction of CTGF, Fibronectin, and Collagen Type IV

The ability of statins to inhibit TGF-β1-mediated induction of CTGF, fibronectin, and collagen type IV was assessed in HDF cells. In this experiment, cells were treated with the indicated amount of simvastatin in the presence of 10 ng/ml of TGF-β1 for 20 hours. In order to determine if simvastatin affects expression of CTGF and fibrotic proteins mRNAs, RNA was extracted from HDF cells treated with both TGF-β1 and simvastatin, and RT-PCR was performed to specifically detect expression of CTGF, fibronectin (FN), collagen type IV (Col IV), and hβ₂M mRNA. FIG. 10A is an agarose gel of an RT-PCR analysis of mRNA expression in HDF cells treated with TGF-β1 and treated with simvastatin. In the presence of TGF-β1, but the absence of simvastatin, expression of CTGF, FN, and Col IV mRNA increased relative to control cells, similar to that observed in FIG. 9A. Upon the addition of 0.1 μM simvastatin, a reduction in CTGF, FN, and Col IV mRNA expression was observed. As the concentration of simvastatin increased, the reduction in CTGF, FN, and Col IV mRNA expression became more pronounced. To control for the RT-PCR process, expression of hβ₂M mRNA was used as a control.

Protein expression in HDF cells treated with both TGF-β1 and simvastatin was also determined. Cells were treated with the indicated amount of simvastatin in the presence of 10 ng/ml of TGF-β1 for three (3) days. Following treatment with both simvastatin and TGF-β1, HFL-1 cells were harvested, and cell lysates were separated by SDS-PAGE. The separated proteins were then transferred to nitrocellulose, and Western immunoblotting was performed with antibodies specific for CTGF, fibronectin, and α-tubulin. FIG. 10B is a Western blot of HFL-1 cells treated with both TGF-β1 and simvastatin. Similar to the results of mRNA expression in FIG. 10A, the amount of CTGF detected in both the culture medium (CM) and the cell lysate (CL) decreased as the amount of simvastatin increased. A decrease in protein expression of fibronectin was also observed. As a control for protein loading, α-tubulin expression in HFL-1 cells was also determined.

### Example 8

### Induction of CTGF, Fibronectin, and Collagen Type IV with Radiation

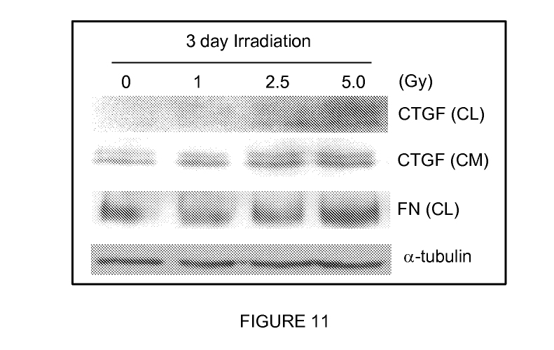

In an effort to determine the mechanism of radiation-induced fibrosis, HDF cells were exposed to increasing amounts of radiation, ranging from 0 to 5 Gy. Three days following exposure to the indicated amount of radiation, HDF cells were harvested, and cell lysates were separated by SDS-PAGE. The separated proteins were then transferred to nitrocellulose, and Western immunoblotting was performed with antibodies specific for CTGF, fibronectin, and α-tubulin. FIG. 11 is a Western blot of HDF cells exposed to radiation. As shown in FIG. 11, the amount of CTGF in both the culture medium (CM) and the cell lysate (CL) increased as the amount of radiation increased. The same increase in protein expression was also observed for fibronectin. As a control for protein loading, α-tubulin expression in HFL-1 cells was also determined.

What is claimed is:

1. A method for treating or preventing radiation-induced fibrosis, comprising, administering to a human or animal, an effective amount of a composition that inhibits CTGF.

2. The method of claim 1, wherein the composition that inhibits CTGF is administered to the human or animal prior to exposure of the human or animal to ionizing radiation.

3. The method of claim 1, wherein the composition that inhibits CTGF is administered to the human or animal concurrently with exposure of the human or animal to ionizing radiation.

4. The method of claim 1, wherein the composition that inhibits CTGF is administered to the human or animal after the human or animal is exposed to ionizing radiation.

5. The method of claim 1, wherein the composition comprises at least one HMG-CoA reductase inhibitor compound.

6. The method of claim 1, wherein at least one HMG-CoA reductase inhibitor compound is compactin, lovastatin, simvastatin, pravastatin, fluvastatin, atorvastatin, mevastatin, rivastatin, cer(i)vastatin, pitavastatin, nisvastatin, itavastatin, rosuvastatin, berivastatin, dalvastatin, glenvastatin, dihydromevinolin, visastatin or combinations thereof.

7. The method of claim 6, wherein at least one HMG-CoA reductase inhibitor compound is simvastatin.

8. The method of claim 1, wherein the composition comprises CTGF inhibitors comprising antibodies that bind to at least a portion of CTGF, antisense molecules comprising CTGF sequences, humanized antibodies that bind CTGF, antibody fragments or active sites that bind at least a portion of CTGF, interfering peptides, interfering nucleic acids, siRNA, antisense RNA, or nucleic acids that interfere with or prevent the transcription or translation of CTGF genes.

9. A method for treating or preventing a pathology related to CTGF, comprising, administering to a human or animal an effective amount of a composition that inhibits CTGF activity.

10. The method of claim 9, wherein the pathology related to CTGF is radiation-induced fibrosis.

11. The method of claim 9, wherein the composition that inhibits CTGF comprises at least one HMG-CoA reductase inhibitor compound, a combination of HMG-CoA reductase inhibitor compounds, antibodies that bind to at least a portion of CTGF, antisense molecules comprising CTGF sequences, humanized antibodies that bind CTGF, antibody fragments or active sites that bind at least a portion of CTGF, interfering peptides, interfering nucleic acids, siRNA, antisense RNA, or nucleic acids that interfere with or prevent the transcription or translation of CTGF genes.

12. The method of claim 11, wherein the composition that inhibits CTGF comprises at least one HMG-CoA reductase inhibitor compound.

13. The method of claim 12, wherein at least one HMG-CoA reductase inhibitor compound is simvastatin.

14. A method for treating neoplastic disease, comprising,
administering an effective amount of a composition that inhibits CTGF to a human or animal, and
irradiating the human or animal with an effective amount of ionizing radiation to affect the neoplastic disease.

15. The method of claim 14, wherein the composition that inhibits CTGF is administered to the human or animal prior to exposing the human or animal to ionizing radiation.

16. The method of claim 14, wherein the composition that inhibits CTGF is administered to the human or animal concurrently with exposing the human or animal to ionizing radiation.

17. The method of claim 14, wherein the composition that inhibits CTGF is administered to the human or animal after the human or animal is exposed to ionizing radiation.

18. The method of claim 14, wherein the composition that inhibits CTGF activity comprises at least one HMG-CoA reductase inhibitor compound, a combination of HMG-CoA reductase inhibitor compounds, antibodies that bind to at least a portion of CTGF, antisense molecules comprising CTGF sequences, humanized antibodies that bind CTGF, antibody fragments or active sites that bind at least a portion of CTGF, interfering peptides, interfering nucleic acids, siRNA, antisense RNA, or nucleic acids that interfere with or prevent the transcription or translation of CTGF genes.

19. The method of claim 14, wherein the composition that inhibits CTGF comprises at least one HMG-CoA reductase inhibitor compound.

20. The method of claim 19, wherein at least one HMG-CoA reductase inhibitor compound is simvastatin.


SEQUENCE LISTING (8 sequences)

SEQ ID NO: 1 - DNA, 20 bp, Artificial Sequence
  CTGF forward primer
ctggtccagaccacagagtg

SEQ ID NO: 2 - DNA, 20 bp, Artificial Sequence
  CTGF reverse primer
cggtatgtcttcatgctggt

SEQ ID NO: 3 - DNA, 20 bp, Artificial Sequence
  COL-IV forward primer
agcaaggcaacagaggactt

SEQ ID NO: 4 - DNA, 20 bp, Artificial Sequence
  COL-IV reverse primer
gatctgggtggaaggtgact

SEQ ID NO: 5 - DNA, 20 bp, Artificial Sequence
  FN forward primer
gactggagctggagacatga

SEQ ID NO: 6 - DNA, 20 bp, Artificial Sequence
  FN reverse primer
gtgatgatggtggactgctc

SEQ ID NO: 7 - DNA, 17 bp, Artificial Sequence
  hB2M forward primer
gtgctcgcgctctctct

SEQ ID NO: 8 - DNA, 20 bp, Artificial Sequence
  hB2M reverse primer
cggcaggcatactcatcttt